(19) United States
(12) Patent Application Publication
(10) Pub. No.: US 2004/0098000 A1
(43) Pub. Date: May 20, 2004

(54) Surgical saw blade

(75) Inventor: Timo Kleinwaechter, Tuttlingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/615,311

(22) Filed: Jul. 7, 2003

(30) Foreign Application Priority Data
Jul. 8, 2002 (DE) 102 31 393

(51) Int. Cl. A61B 17/32

(52) U.S. Cl. 606/167

(57) ABSTRACT

In order to improve a surgical saw blade comprising a holder body and a row of teeth which includes a plurality of teeth and is arranged at one end of the holder body wherein each tooth is formed with three flanks in the vicinity of the tip of a tooth such that it will be utilisable in a simple and reliable manner, it is proposed that there be formed between adjacent teeth a channel via which, and in relation to the tip of a tooth, the cuttings of material are adapted to be carried away to behind the row of teeth.

The present disclosure relates to the subject matter disclosed in German application No. 102 31 393.8 of Jul. 8, 2002, which is incorporated herein by reference in its entirety and for all purposes.

### BACKGROUND OF THE INVENTION

The invention relates to a surgical saw blade comprising a holder body and a row of teeth which incorporates a plurality of teeth and is arranged at one end of the holder body, wherein each tooth is formed with three flanks in the vicinity of the tip of a tooth.

Surgical saw blades of this type are utilised especially in oscillating saws for sawing bones during orthopaedic operations for example.

A saw blade, which comprises non-crossed teeth whose lateral faces are parallel to the lateral faces of the saw blade, is known from DE 32 22 339 C2.

A saw blade comprising teeth that exhibit a so-called diamond cut is known from U.S. Pat. No. 5,306,285.

### SUMMARY OF THE INVENTION

In accordance with the present invention, a surgical saw blade is improved such that it will be utilisable in a simple and reliable manner. In accordance with the invention, there is formed between adjacent teeth a channel via which, and relative to the tooth tip, the cuttings of material are adapted to be carried away behind the row of teeth.

Saw blades comprising teeth which are formed with three flanks in the vicinity of the tip of a tooth and especially ones exhibiting a diamond cut, namely teeth which comprise tooth flanks that are at an angle relative to the upper or lower face of the saw blade have the advantage that a high performance sawing action is achievable therewith without the need to cross these teeth.

Consequently, the saw blade can also be fed through a slot in a template such as is employed in the case of knee operations for example. Improved guidance in a saw slot and hence a reduction in the tendency of the saw blade to run untrue can be achieved thanks to the provision of enlarged guide surfaces by means of appropriate tooth flanks in the row of teeth.

Very thin layers of bone can also be removed by means of a diamond cut.

Due to the provision of channels between adjacent teeth in accordance with the invention, the cuttings of material produced during the sawing process can be removed from the operative zone of the saw thus ensuring highly efficient removal of these cuttings of material even in the case of triple-flanked teeth and especially those with a diamond cut despite the larger guide surface in the saw slot. This improved removal process is achieved without having to cross the teeth. Thus, in accordance with the invention, improved removal of the cuttings of material is achieved, whereby nevertheless, a large guide surface for the teeth is also achieved.

Consequently, precisely cut slots can be produced, namely, the danger of the saw blade running untrue is minimised and the roughness of the cut surface is reduced since the danger of the large surfaced tooth flanks penetrating laterally into the material of the bone is also reduced.

In particular, it is advantageous if the channel extends behind a tooth base of the adjacent teeth. Bone cuttings can thereby be removed from the operative zone of the saw blade in a saw slot into the region behind the row of teeth.

Furthermore, it is expedient if the channel extends behind the tooth base at a height which lies within a range of between 20% and 60% of the height of the tooth above the tooth base. Effective removal of the cuttings of material from the operative zone is then attained in this manner.

In an embodiment that is expedient for manufacturing reasons, the channel is in the form of a trough or a notch and is arranged between adjacent teeth in order to ensure effective removal of the cuttings.

In connection with teeth having triple flanks at least in the vicinity of the tip of the tooth, it is advantageous for the channel to be formed between opposed, non-parallel tooth flanks of the adjacent teeth, namely, especially in the case of teeth having a diamond cut, for it to be formed in the region between the tooth flanks which are at an angle relative to an upper face or a lower face of the holder body.

It is particularly advantageous if the channels between adjacent teeth are connected to at least one holder body channel which is formed in the holder body. Due to this arrangement, cuttings of material can then flow away via the channels into the holder body channel in order to enable the cuttings of material to be carried away from the row of teeth i.e. away from the operative zone of the saw blade in a saw slot.

In particular hereby, said at least one holder body channel may be arranged behind the tooth base in order to ensure effective removal of the cuttings.

Furthermore, it is expedient if said at least one holder body channel extends along the row of teeth so that each of the channels between adjacent teeth is adapted to be fed into the holder body channel. This also simplifies the manufacture of the saw blade in accordance with the invention. Hereby, provision may be made for a channel to be formed between selected adjacent teeth, although it is particularly advantageous if a respective channel is formed between each of the adjacent teeth so that all of these channels then lead into the at least one holder body channel.

Furthermore, it is expedient if, in the direction towards the row of teeth, said at least one holder body channel is bounded thereby, this thus simplifying manufacture on the one hand and minimising the return flow of the cuttings of material from the holder body channel towards the tips of the teeth on the other.

It is particularly expedient if a respective holder body channel is formed on a lower face and on an upper face of the holder body so that the cuttings of material can flow away from the row of teeth over both sides of the holder body. Particularly effective removal of the cuttings of material from the operative zone is achieved in this manner.

It is expedient hereby if provision is made for the two holder body channels to be substantially parallel to one another i.e. parallel to one another on opposite sides of the holder body, in order to ensure that the cuttings of material are removed in a uniform manner and far as possible without clogging.

It has also proved to be advantageous if the depth of the at least one holder body channel with reference to a thickness of the holder body (in the same direction of separation) lies within a range of between 15% and 35% of this thickness, and especially approximately 20% to 25% of this thickness. Hereby, with respect to the upper face and/or the lower face of the holder body, such a holder body channel is preferably in the form of a depression that is formed therein.

In order to obtain uniform guidance of the row of teeth using a large guide surface in a saw slot, a first tooth flank is substantially parallel to an upper face and/or a lower face of the holder body.

Preferably hereby, the first tooth flanks of adjacent teeth are parallel to one another whereby the tooth flanks of the next but one adjacent teeth lie in a plane so that two planes are obtained in this manner, these planes being displaced relative to one another in a direction of separation (width) of the holder body and thus being mutually parallel. The aforementioned large guide surface for the introduction of the cut can then be achieved thereby.

Furthermore, it is then expedient if further second tooth flanks and third tooth flanks are arranged at an angle relative to an upper face and a lower face of the holder body so as to obtain a diamond cut having the above-mentioned advantages.

The tooth tips of adjacent teeth are preferably displaced relative to one another with reference to a direction of separation of the holder body and, in particular hereby, they are displaced alternately i.e. the tooth tips of adjacent teeth are spaced in the direction of separation of the holder body. A large guide surface is therefore obtained on each side in the saw slot by virtue of the parallel tooth flanks of adjacent teeth. Point like contact with the bone material in the saw slot is thereby avoided in the operative zone of the row of teeth. Due to the channels in accordance with the invention, highly efficient removal of the cuttings of material from the operative zone is thereby attained.

The holder body has a flat construction and it preferably extends substantially equidistantly between a first surface and a second surface. A slotted template can thereby be used in order to enable a spatially defined, precise cut to be made in the bone material.

It is particularly expedient if, at least in the vicinity of the tooth tip, the first tooth flank protrudes beyond the associated first surface or the associated second surface taken with reference to a direction of separation of the holder body. A saw slot can thereby be produced which is wider than that which corresponds to the thickness of the holder body. This means, especially in connection with a slotted template guidance arrangement, that only the row of teeth will come into direct frictional contact with the bone material in the saw slot. The holder body itself does not come into frictional contact (apart from the indirect contact through the cuttings of material) so that the development of heat in the saw slot is reduced, i.e. the frictional surface is minimised even in the case of large guide surfaces due to the first tooth flanks. In turn, the danger of heat necrosis, which could lead to the danger of the healing process not being successful, is thereby reduced. Due to the reduced friction, a higher cutting performance can be obtained in conjunction with an oscillating saw using a rechargeable battery for the same size of battery.

Hereby, it is particularly advantageous if the first tooth flank is displaced substantially parallel relative to the first surface and the second surface i.e. if the teeth are not set even though the row of teeth projects out beyond the holder body. The advantages mentioned above in connection with the diamond cut can thus be achieved i.e. a relatively large guidance surface, but wherein the effective frictional surface of the saw blade in the holder body is simultaneously minimised, in accordance with the invention, due to the projection of the teeth beyond the holder body.

It has proved to be expedient if the thickness of the row of teeth is between 4% and 12%, and especially approximately 7%, more than the spacing between the first surface and the second surface.

Furthermore, it is then expedient if the first tooth flanks of the next but one adjacent teeth in the row of teeth lie in a plane so that a corresponding large guide surface for guiding the teeth in the saw slot is provided on both sides of the row of teeth.

In order to enable the surgical saw blade to be fixed to an oscillating saw, a receiving portion (seating) for fixing the saw blade is formed in an end area of the holder body that is remote from the row of teeth.

It has also proved to be expedient if the holder body comprises a resilient portion and a stiff portion, whereby the flexural rigidity of the resilient portion is lower than that of the stiff portion which supports the row of teeth. In particular, if a saw blade is guided by means of a slotted template, then it may happen that a slight axial error of alignment of the saw blade may be present. Such an axial error of alignment should not manifest itself in the form of an error of alignment of the annexed part of the saw blade on the bone. If a resilient portion is provided, especially one that follows a seating portion which incorporates the seating used for the purposes of fixing it the oscillating saw, then such an axial error of alignment will be corrected without adversely affecting the results of the sawing process. The increased stiffness of the stiff portion carrying the row of teeth ensures that a precise saw slot can be produced whereby even the portions relevant for the sawing process (the rows of teeth) are not bent. (The actual error of alignment relates to an error of alignment of the seating portion of the saw blade relative to a slot in the slotted template.)

It has also proved to be expedient if a plurality of channel-like recesses, especially in the form of depressions, are arranged in the upper face and/or the lower face of the holder body. The cuttings of material can be removed via these recesses on the one hand. On the other hand, the total mass of the saw blade can thereby be reduced and thus the efficiency of the sawing process can be increased in the case of rechargeable battery operation for a similar battery power. Furthermore, the proportion of potential zones of friction will thereby be reduced (also in connection with the removal of the cuttings of material) and, in consequence, the efficiency of the sawing process will thus be increased. Simultaneously hereby, the thermal load on the bone will, in turn, be reduced.

In particular hereby, the recesses are formed symmetrically with reference to an axis of symmetry extending between the opposed sides of the holder body so as not to produce unbalance with regard to the oscillation of the saw blade.

It is expedient if the stiff portion and the resilient portion are produced by means of the arrangement and construction of the recesses. For example, an increased stiffness of the stiff portion can be achieved if the recesses are in the form of a parabola i.e. the walls of the recesses that bound the recesses have a parabolic shape.

The following description of a preferred embodiment will, in conjunction with the drawing, serve to explain the invention in more detail.

### BRIEF DESCRIPTION OF THE DRAWINGS

FIG. 1 shows a plan view of an embodiment of a saw blade in accordance with the invention;

FIG. 2 a perspective partial view of a row of teeth on the saw blade of FIG. 1;

FIG. 3 an enlarged view of the area A in FIG. 2;

FIG. 4 a partial sectional view along the line 4-4 in FIG. 1, and

FIG. 5 a schematic illustration of a saw slot that has been sawn out by a saw blade in accordance with the invention together with a partial view of the saw blade dipping into the saw slot.

### DETAILED DESCRIPTION OF THE INVENTION

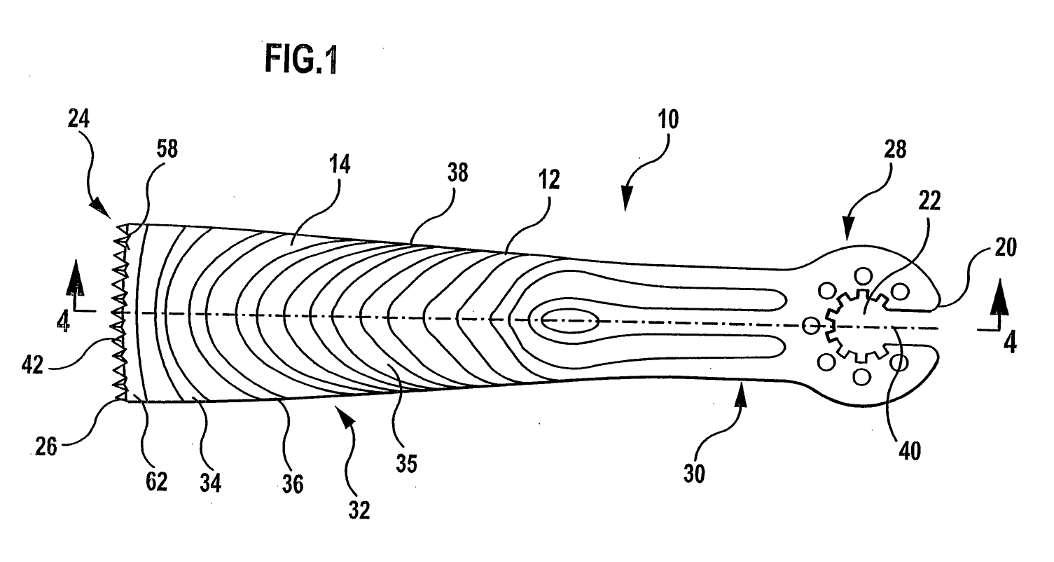

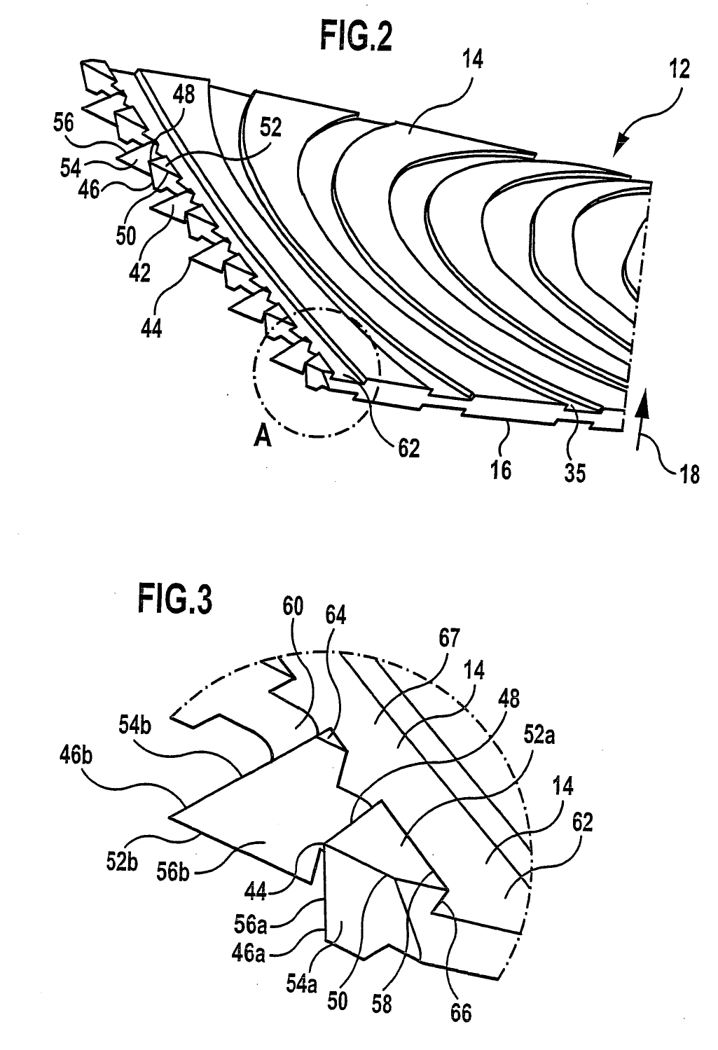

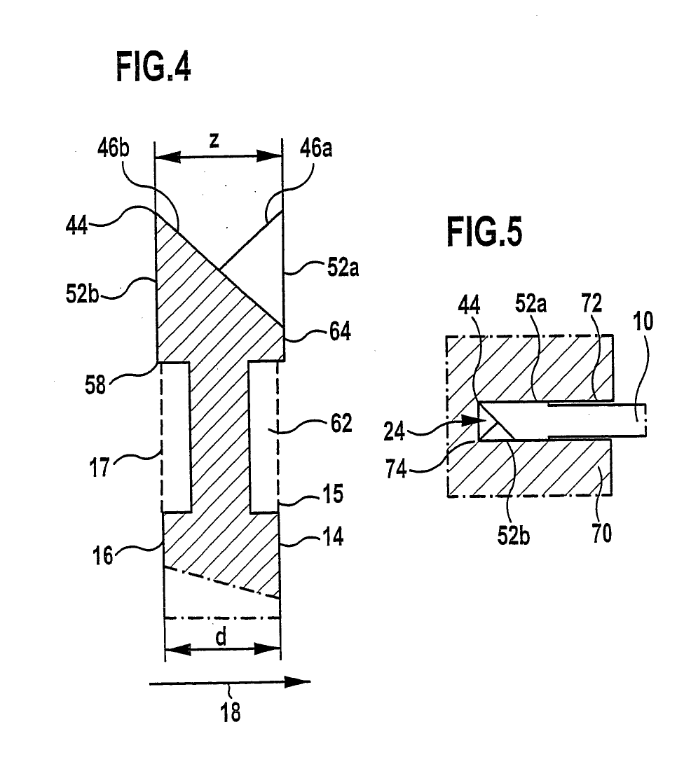

Surgical saw blades are used in conjunction with oscillating saws especially for orthopaedic purposes. In an embodiment of a saw blade in accordance with the invention which bears the general reference 10 in FIG. 1, there is provided a holder body 12 having an upper face 14 and a lower face 16 (FIG. 2) which extends between substantially parallel surfaces 15 and 17 (FIGS. 2 and 4). The spacing d between these surfaces 15 and 17 in the direction of separation 18 (direction of width) perpendicular thereto is substantially constant and represent the thickness d of the holder body 12. A typical value for this thickness d is 1.37 mm.

The holder body 12 is flat i.e. the width of the holder body 12 transverse to the direction of separation 18 is considerably greater than the thickness d in the direction of separation 18. The length of the holder body 12 is likewise considerably greater than the thickness d. A typical value for the length is approximately 110 mm.

In the vicinity of its one end 20, the holder body 12 is provided with a seating 22 (receiving portion) with the aid of which the surgical saw blade 10 is adapted to be fixed to an oscillating saw in order to enable an oscillatory movement, which is produced by a driver device in the oscillating saw, to be converted into a corresponding sawing movement of a row of teeth 24 which is arranged at an end 26 of the holder body 12 remote from the end 20.

The holder body 12 itself comprises a seating area 28 in which the seating 22 is arranged, an adjoining resilient portion 30 and a stiff portion 32 which adjoins this resilient portion 30. The row of teeth 24 is arranged on the stiff portion 32.

Commencing from the resilient portion 30, the holder body 12 widens continuously towards the end 26 of the stiff portion 32 so that it is at its widest at the end 26. The holder body 12 likewise becomes wider at the point of transition between the resilient portion 30 and the seating portion 28 in order to provide a sufficiently wide area for the seating 22.

The flexural rigidity of the resilient portion of the holder body 12 is less than that of the stiff portion 32. Due to the lower stiffness of the resilient portion 30, it is therefore ensured that a slight axial error of alignment with reference to a slot-like template is correctable without the cutting parts of the saw blade 10, namely the row of teeth 24, being canted whilst making a saw cut. A precision cut can therefore be produced despite such an error of alignment.

The difference in flexural rigidity between the stiff portion 32 and the resilient portion is created by a different arrangement and construction of recesses 34 which are formed in the upper face 14 and the lower face 16 of the holder body 12 as depressions having setback recess-bases 35 (the thickness d of the holder body 12 is measured outside these recesses 34).

The recesses 34 in the stiff portion 32 of the holder body 12 extend between the opposed sides 36 and 38 of the holder body 12. They are formed in such a manner that the cuttings of material can be guided away thereby. The mass of the saw blade 10 can be reduced by virtue of the recesses 34, and the effective surface area, which is responsible for the frictional effect within the template and within the saw slot, can be reduced and consequently, this friction will also be reduced. Hereby, the recesses 34 are symmetrical especially with reference to a longitudinal direction 40 of the holder body 12, and they are arranged in parabolic manner in the stiff portion 32 in order to increase the stiffness thereof in comparison to that in the resilient portion 30.

The row of teeth 24 comprises a plurality of tetrahedral teeth 42 wherein each tooth 42 has a tooth tip 44 (FIG. 2).

A first tooth edge 46, a second tooth edge 48 and a third tooth edge 50 meet together at the tooth tip 44. These teeth edges 46, 48, 50 are formed, in turn, by the lines of impingement between a first tooth flank 52, a second tooth flank 54 and a third tooth flank 56 (FIG. 2).

The tooth tips 44 of adjacent teeth 42 in the row of teeth 24 are mutually displaced in relation to the direction of separation 18, whereby the first tooth flanks 52 of the next but one adjacent teeth 42 lie substantially in a plane i.e. the teeth 42 are not set.

The tooth flanks 54 and 56 are at an angle relative to the plane included by the first tooth flanks 52 of the next but one adjacent teeth 52 and they intersect along the first tooth edge 46 which is at an angle relative to this plane.

The first tooth edges 46 of adjacent teeth 42 (referenced by 46a and 46b in FIG. 3) are disposed at an angle relative to one another i.e. they are not parallel whereby, with reference to the upper face 14 for example, the first tooth edge 46a is at an acute angle relative to this upper face 14 whilst the adjacent tooth edge 46b forms an obtuse angle therewith and the two angles differ by 90° (FIG. 4).

A tooth construction of this type comprising tetrahedral teeth 42 having parallel tooth flanks 52 at least in the vicinity of the tooth tips 44 is also referred to as a diamond cut.

The opposed second tooth flanks 54a, 54b of adjacent teeth 42 and the adjacent third tooth flanks 56a, 56b of adjacent teeth 42 are likewise disposed at an angle relative to one another (FIG. 3).

The first tooth flanks 52a, 52b of adjacent teeth 42 are mutually parallel.

Hereby, the teeth 42 extend from a tooth base 58 up to the respective tooth tip 44. This tooth base forms the base surface for the teeth 42 via which they are connected to the holder body 12.

A respective channel 60, via which the cuttings produced during the cutting process are adapted to be removed, is formed between adjacent teeth 42. Such a channel 60 is in the form of a trough or notch and is arranged between opposed second tooth flanks 54a, 55b or between opposed tooth flanks 56a, 56b of adjacent teeth 42. Hereby, the channels 60 extend away from the tooth tip 44 beyond the tooth base 58 at a depth of between 20% and 60% of the height of the tooth above the tooth base 58 so that the cuttings are adapted to be fed away from the tooth tip 44 to a point behind the tooth base 58.

Hereby, the channels 60 of the row of teeth 24 lead to a holder body channel 62 which is formed in the holder body 12 behind the tooth base 58 as a recess in the form of a depression. The cuttings can thereby be fed away from the tooth tip 44 to a point behind the tooth base 58 in order to effect the removal of the cuttings. The holder body channel 62 extends along the row of teeth 24 and is bounded by the tooth base 58 in the direction towards the tooth tips 44.

A typical depth measurement (taken with respect to the direction of separation 18) for the holder body channel 62 from the upper face 14 or the lower face 16 is between 15% and 35% of the thickness d of the holder body 12 and is preferably approximately 20% of the thickness d. A typical value for this channel depth is 0.33 mm in the case of a thickness d of 1.37 mm.

If the first tooth edges 46 are arranged at an angle such that they do not reach as far as the tooth base 58 (FIG. 4), then a tooth flank 64 is formed between the tooth base 58 and the first tooth edge 46 in parallel with and especially in the plane of the corresponding first tooth flanks 52a or 52b of adjacent teeth 42 so that this tooth flank 64 does not protrude beyond the respective plane of the first tooth surfaces 52. In the case of such a construction, a tooth comprises five sides whereby however, three tooth edges 46, 48, 50 meet together at the tooth tip 44. The tooth 42 is tetrahedral in the vicinity of the tooth tip 44. The overall shape corresponds to a tetrahedron from which a (smaller) tetrahedron has been cut out.

The holder body 12 extends equidistantly between the first surface 15 and the second surface 17, namely, the thickness d (FIG. 4). The spacing z between the tooth tips 44 of adjacent teeth 42 in the direction of separation 18 is greater then this distance d, whereby the difference in these magnitudes lies in a range of between 4% and 12% and is especially, approximately 7%. If, for example, the thickness d is 1.37 mm, then a preferred value for the thickness z of the row of teeth 24 is then 1.47 mm.

This mean that the first tooth flanks 52a or 52b are parallel with but offset from the first surface 15 or the second surface 17. Thus, with reference to the corresponding first surface 15 or the second surface 17 (in dependence upon whether the tooth tip 44 is closer to the lower face 16 or the upper face 14), the tooth tips 44 are spaced therefrom by a certain amount in the direction of separation 18; this is 0.05 mm in the aforementioned numerical example. It is thereby possible to produce a saw slot which is wider in the direction of separation 18 than the width corresponding to the thickness of the holder body 12.

Hereby, a boundary surface 66 of the tooth base 58 also bounds the holder body channel 62 at the side next to the row of teeth 24, namely in the region wherein the tooth 42 protrudes above a channel base 67 which is deepened with respect to the surface 15 or 17.

The saw blade 10 in accordance with the invention functions as follows:

The saw blade 10 is clamped in an oscillating saw and is fed towards the bone 70 that is to be sawn (FIG. 5) via a template providing slot guidance. Due to the oscillating movement, a cut is made in the bone 70 and a saw slot 72 is thereby produced. The resultant bone cuttings are fed away from the row of teeth 24 via the respective channels 60 and the holder body channels 62 in the lower face 16 and upper face 14 of the holder body. The further recesses 34 also assist in the removal of the cuttings of material.

Taken with reference to the direction of separation 18, the saw blade 10 has a greater thickness z in the vicinity of the row of teeth 24 than it has in the vicinity of the holder body 12 (thickness d). A saw slot 72 is thereby produced during the sawing process which is wider than the holder body 12 in the direction of separation 18. As a result, for the purposes of enlarging the saw slot 72, a guide surface formed by the respective first tooth flanks 52a, 52b is fed into the saw slot 72 during the sawing process whereby, in turn, the guidance of the saw blade 10 in the saw slot 72 is improved. This then improves the smoothness of the cut surface in the saw slot 72.

Furthermore, since the holder body 12 does not touch the walls of the saw slot 72, friction in the saw slot 72 is reduced and consequently the effectiveness of the sawing process is increased. Furthermore, the thermal load on the bone 70 is reduced and hence the danger of heat necrosis is reduced.

At the same time, highly efficient removal of the cuttings from an operative zone 74 of the row of teeth 24 is ensured.

1. A surgical saw blade comprising:
a holder body;
a row of teeth having a plurality of teeth and being arranged at one end of the holder body;
wherein each tooth is formed with three flanks in the vicinity of the tip of a tooth; and
a channel being formed between adjacent teeth via which the cuttings of material are adapted to be carried away to behind the row of teeth in relation to the tooth tip.

2. A surgical saw blade in accordance with claim 1, wherein the channel extends behind a tooth base of the adjacent teeth.

3. A surgical saw blade in accordance with claim 2, wherein the channel extends behind the tooth base at a height which lies within a range of between 20% and 600/% of the height of the tooth above the tooth base.

4. A surgical saw blade in accordance with claim 1, wherein the channel is in the form of a trough.

5. A surgical saw blade in accordance with claim 1, wherein the channel is formed between opposed, non-parallel tooth flanks of adjacent teeth.

6. A surgical saw blade in accordance with claim 1, wherein the channels between adjacent teeth are connected to at least one holder body channel which is formed in the holder body.

7. A surgical saw blade in accordance with claim 6, wherein said at least one holder body channel is arranged behind the tooth base.

8. A surgical saw blade in accordance with claim 6, wherein said at least one holder body channel extends along the row of teeth.

9. A surgical saw blade in accordance with claim 6, wherein, in the direction towards the row of teeth, said at least one holder body channel is bounded by said row of teeth.

10. A surgical saw blade in accordance with claim 6, wherein a respective holder body channel is formed on a lower face and on an upper face of the holder body.

11. A surgical saw blade in accordance with claim 10, wherein the two holder body channels are substantially parallel to one another.

12. A surgical saw blade in accordance with claim 6, wherein the depth of said at least one holder body channel with reference to a thickness of the holder body lies within a range of between 15% and 35% of this thickness.

13. A surgical saw blade in accordance with claim 1, wherein a first tooth flank is substantially parallel to at least one of an upper face or a lower face of the holder body.

14. A surgical saw blade in accordance with claim 13, wherein the first tooth flanks of adjacent teeth are parallel to one another.

15. A surgical saw blade in accordance with claim 13, wherein further second tooth flanks and third tooth flanks are arranged at an angle to an upper face and a lower face of the holder body.

16. A surgical saw blade in accordance with claim 1, wherein the tooth tips of adjacent teeth are displaced relative to one another with reference to a direction of width of the holder body.

17. A surgical saw blade in accordance with claim 1, wherein the holder body extends substantially equidistantly between a first surface and a second surface.

18. A surgical saw blade in accordance with claim 17, wherein, at least in the vicinity of the tooth tip, the first tooth flank protrudes beyond the associated first surface or the associated second surface taken with reference to a direction of width of the holder body.

19. A surgical saw blade in accordance with claim 18, wherein the first tooth flank is displaced substantially parallel relative to the first surface or the second surface.

20. A surgical saw blade in accordance with claim 18, wherein a thickness in the area of the row of teeth between first tooth flanks of adjacent teeth is between 4% and 12% more than the spacing between the first surface and the second surface.

21. A surgical saw blade in accordance with claim 1, wherein the first tooth flanks of the next but one adjacent teeth in the row of teeth lie in a plane.

22. A surgical saw blade in accordance with claim 1, wherein a receiving portion for fixing the saw blade in an oscillating saw is formed in an end area of the holder body that is remote from the row of teeth.

23. A surgical saw blade in accordance with claim 1, wherein the holder body comprises a resilient portion and a stiff portion, whereby the flexural rigidity of the resilient portion is lower than that of the stiff portion which supports the row of teeth.

24. A surgical saw blade in accordance with claim 1, wherein a plurality of channel-like recesses are arranged at least in one of the upper face or the lower face of the holder body.

25. A surgical saw blade in accordance with claim 24, wherein the recesses are formed symmetrically with reference to an axis of symmetry extending between the opposed sides of the holder body.

26. A surgical saw blade in accordance with claim 23, wherein the stiff portion and the resilient portion are produced by means of the arrangement and construction of the recesses.

27. A surgical saw blade in accordance with claim 1, wherein a thickness in the row of teeth is greater than the thickness outside the row of teeth on the holder body.
